(19) United States
(12) Patent Application Publication
(10) Pub. No.: US 2013/0331442 A1
(43) Pub. Date: Dec. 12, 2013

(54) Combination Therapy for Cancer

(71) Applicants: Haritha SAMARANAYAKE, Kuopio (FI); Jere PIKKARAINEN, Kuopio (FI); Ann-Marie MAATTA, Kuopio (FI); Seppo YLA-HERTTUALA, Kuopio (FI)

(72) Inventors: Haritha SAMARANAYAKE, Kuopio (FI); Jere PIKKARAINEN, Kuopio (FI); Ann-Marie MAATTA, Kuopio (FI); Seppo YLA-HERTTUALA, Kuopio (FI)

(73) Assignee: FINVECTOR VISION THERAPIES LTD, Oxfordshire (GB)

(21) Appl. No.: 13/966,376

(22) Filed: Aug. 14, 2013

Related U.S. Application Data
(63) Continuation-in-part of application No. 13/858,393, filed on May 1, 2013.
Application No. 13/858,393 is a continuation-in-part of application No. 13/877,246, filed on Aug. 7, 2013.

(30) Foreign Application Priority Data
Jan. 18, 2011 (GB) GB1100804.2
Jan. 18, 2012 (GB) PCT/GB2012/050108

(51) Int. Cl. A61K 31/7088; A61K 31/522; A61N 5/00; A61K 31/495

(52) U.S. Cl. CPC A61K 31/7088; A61K 31/495; A61K 31/522; A61N 5/00
USPC 514/44 R; 600/1

(57) ABSTRACT

An agent comprises a vector having a functional gene, a prodrug which can be converted into a cytotoxic agent by an expression product of the gene, and another cytotoxic agent, as a combined preparation for simultaneous, sequential or separate use in the therapy of cancer or of a disease characterised by an impaired mismatch repair (MMR) pathway, wherein the dosage regimen comprises beginning another cytotoxic agent therapy no later than 7 days after the prodrug therapy has finished.

### RELATED APPLICATIONS

This application is a continuation-in-part application of U.S. application Ser. No. 13/858,393, filed on May 1, 2013, which is a continuation-in-part application of U.S. application Ser. No. 13/877,246, filed on Apr. 1, 2013, which is a US National Stage Application of International Application No. PCT/GB2012/050108, filed Jan. 18, 2012; which claims priority from Great Britain Application Serial No. 1100804.2, filed Jan. 18, 2011; all of which are incorporated herein by reference in their entirety.

### FIELD OF THE INVENTION

This invention relates to a drug combination and method for the treatment of cancer or of a disease characterised by an impaired mismatch repair (MMR) pathway, specifically a drug combination and method for the treatment of glioblastoma multiforme, including recurrent glioblastoma multiforme.

### BACKGROUND OF THE INVENTION

AdHSV-tk/GCV Therapy

Ganciclovir (“GCV”) is a non-toxic compound.

Thymidine kinase is an enzyme coded for by the tk gene. Thymidine kinase converts GCV into a metabolite which is non-toxic for non-dividing cells, yet cytotoxic for dividing cells. This property is of particular advantage in brain cancer therapy, where the rapidly dividing tumour cells are surrounded by and intercalated among non-dividing normal brain cells.

One may transform host cells with the tk gene by using an adenovirus-herpes simplex virus (AdHSV) derived transfection vector: the AdHSV-tk vector transforms host cells so they express the tk gene, making thymidine kinase. Subsequently administered GCV is then converted into the metabolite toxic for dividing cells. Such cell destruction by “AdHSV-tk/GCV” is cell cycle dependent, so only dividing cells (e.g., malignant cells) are affected.

Temozolomide Therapy

Temozolomide (TMZ, imidazole tetrazinone) is an oral alkylating agent that can cross the blood brain barrier (BBB). Temozolomide is an oral alkylating agent that is a derivative of dacarbazine. TMZ undergoes spontaneous hydrolysis at physiological pH to its active form 3-methyl-(triazen-1-yl) imidazole-4 carboxyamide (MTIC). The primary mode of cytotoxicity is by adding a methyl group at the O⁶-position of guanine (O⁶-mG).

O⁶-mG by itself is not toxic to the cells. However, O⁶-mGs will become cytotoxic as a result of repeated cycles of futile efforts at repair by mismatch repair (MMR) pathway. This will ultimately lead to DNA strand breaks. It is known that a functional MMR pathway is essential to make cells sensitive to TMZ, in the absence of an active MGMT repair pathway (which occurs in 50% of malignant gliomas). Furthermore, defects in the MMR pathway can contribute to almost 100-fold resistance to alkylating agents such as TMZ.

### SUMMARY OF THE INVENTION

The present invention is based on our discovery that HSV-tk gene therapy increases the gene expression of key mismatch repair (MMR) pathway proteins, namely MSH2 and MLH1. This led to our finding that HSV-tk/GCV gene therapy sensitises cells to chemotherapeutic agents, such as temozolomide (TMZ).

We designed a study which confirmed that a combination of vector/prodrug gene therapy (such as AdHSV-tk/GCV) and a cytotoxic agent (such as TMZ), has an unexpectedly synergistic efficacy in brain cancer, when compared to the use of either of the components alone, i.e. chemotherapy or vector/prodrug gene therapy.

We also found, somewhat surprisingly, that the administration protocol of these components is critical to the surprising synergistic effect we observed. We found that the up-regulation of the MMR pathway by vector/prodrug gene therapy takes approximately 2 days, and lasts for about 7 days after stopping prodrug therapy. Therefore, in order to see synergy, we found it critical to begin administering the cytotoxic agent during this window of time: likely no later than about 7 days after finishing prodrug therapy, and preferably to begin administering the cytotoxic at the same time one begins to administer the prodrug.

Furthermore, when the condition to be treated is characterised by an impaired MMR pathway, it is believed that a therapeutic benefit may be achieved by administering only the vector/prodrug gene therapy.

In a first aspect, the present invention is characterised by a new dosage regimen. Therefore, according to a first aspect, the present invention is a new dosage regimen combining administration of (1) a vector having a functional gene (e.g., the tk gene), (2) a pro-drug (e.g., GCV) which can be converted into a cytotoxic agent by an expression product of the gene (e.g., thymidine kinase), and (3) a second cytotoxic agent (e.g., TMZ), in a new dosage regimen for use in the therapy of brain cancer, wherein the dosage regimen comprises (1) administering vector; then (2) administering pro-drug; then (3) administering the second cytotoxic agent no later than about 7 days after completing pro-drug therapy.

According to a second aspect, the present invention is an agent comprising a vector having a functional gene, and a pro-drug which can be converted into a cytotoxic agent by an expression product of the gene, as a combined preparation for simultaneous, sequential or separate use in the therapy of a disease characterised by an impaired mismatch repair (MMR) pathway.

### DESCRIPTION OF THE FIGURES

FIG. 1A shows mean tumour volume at day 28 for tumors treated with: Control (saline injection); temazolamide (“TMZ”) for 5 days; Cerepro® brand AdHSV-tk gene therapy vector followed by 6 days of ganciclovir (“GCV”); Cerepro® brand AdHSV-tk gene therapy vector followed by 6 days of GCV followed by a “gap” period (no treatment) of 5 days followed by 5 days of TMZ; and Cerepro® brand AdHSV-tk gene therapy vector followed by 6 days of GCV and by 5 days of TMZ, the first TMZ dose administered on the same day as the last GCV dose.

FIG. 1B shows mean tumour volume at day 42 for tumors treated with the dosage regimens as shown in FIG. 1A.

FIG. 2 shows the Kaplan-Meier survival curves for test subjects administered the dosage regimens as shown in FIG. 1A. FIG. 2 shows that at 50 days, 1 of 9 control subjects survived (black line), 0 of 10 TMZ subjects survived (blue line), and 2 of 19 AdHSV-tk/GCV subjects survived (purple line). FIG. 2 shows that at 50 days, 18% (2 of 11) of GCV+TMZ “gap” subjects survived (red line), while 71% (5 of 7) of GCV+TMZ “bb” (no gap) subjects survived (green line).

FIG. 3 shows relative mRNA expression in cells 3, 5 and 8 days after transformation.

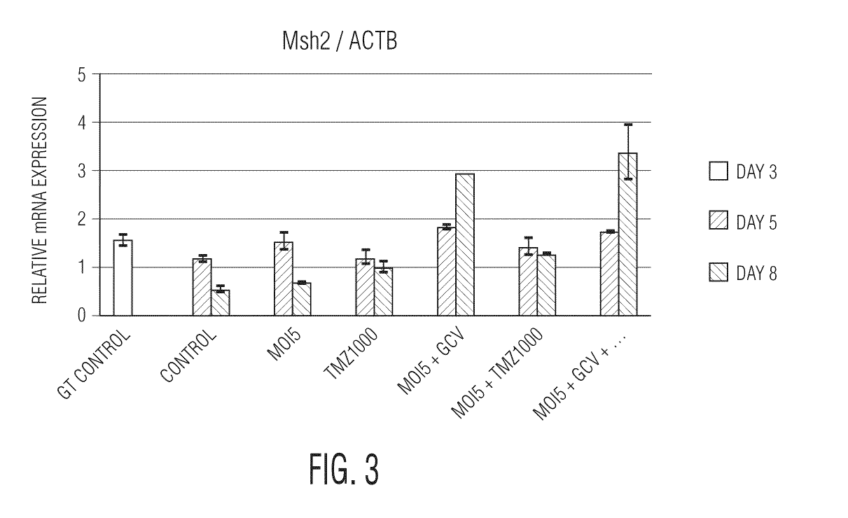

FIG. 4 shows the effect on in vitro cell cultures of different concentrations of TMZ with and without concomitant AdHSV-tk/GCV therapy.

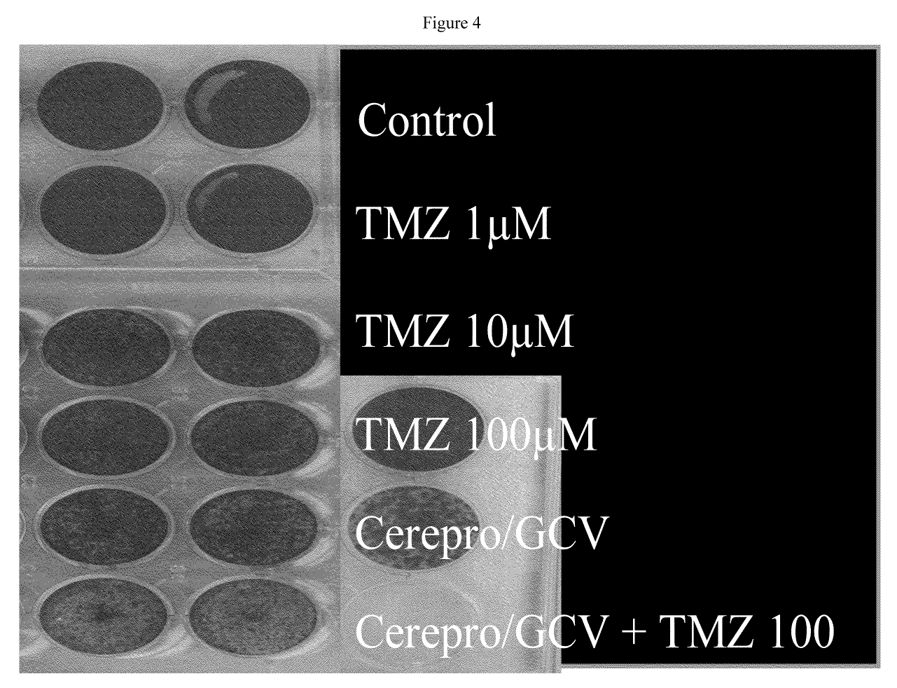

FIG. 5 shows the role of MMR and BER pathways in TMZ induced cytotoxicity.

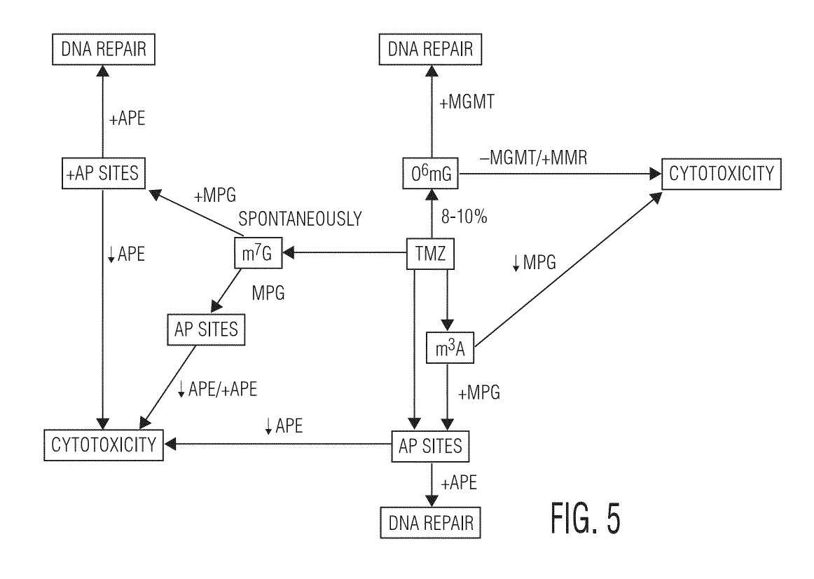

### DETAILED DESCRIPTION

In our AdHSV-tk/GCV in combination with TMZ study, we found that AdHSV-tk/GCV increases the gene expression of key mismatch repair (MMR) pathway proteins, namely MSH2 and MLH1. This finding has not been reported in scientific literature before. Furthermore, the increase in transcription of MMR pathway protein genes was observed 96 h after adding GCV, and the increase persisted for days thereafter. We are explaining the increased cytotoxicity of TMZ in combination with AdHSV-tk/GCV on the basis of this enhancement of MMR pathway by AdHSV-tk/GCV and suggest a change in the treatment protocol to maximise the cytotoxicity when these two therapies are combined.

We believe that, since the enhancement of MMR pathway by AdHSV-tk/GCV has not been reported before, the combination of AdHSV-tk/GCV with TMZ where the cytotoxicity of TMZ is increased by enhancement of MMR pathway is patentable. This will open new doors for AdHSV-tk/GCV (and LentiHSV-tk/GCV) gene therapy in combination with many chemotherapeutic agents in the treatment of several cancers.

Mechanism of Action of TMZ

Temozolomide is an oral alkylating agent derivative of dacarbazine that undergoes spontaneous hydrolysis at physiological pH to its active form 3-methyl-(triazen-1-yl)imidazole-4 carboxyamide (MTIC). The primary mode of cytotoxicity is by adding a methyl group at O⁶-position of guanine (O⁶-mG). A direct repair pathway involving MGMT repairs these lesions, and it is known that cancers that have this functional MGMT repair pathway, are resistant to TMZ therapy. Methylation of this promoter is a known mechanism by which the MGMT pathway is inactivated epigenetically, and is believed to occur in about 50% of malignant gliomas. O⁶-mG by itself is not toxic to the cells. However, these O⁶-mG will become cytotoxic as a result of repeated cycles of futile efforts at repair by MMR pathway. This will ultimately lead to DNA strand breaks (Adhikari et al., 2008). It is known that a functional MMR pathway is needed to make cells sensitive to TMZ, in the absence of an active MGMT repair pathway (Fujio et al., 1989). Furthermore, defects in the MMR pathway can contribute to almost 100-fold resistance to alkylating agents such as TMZ (Papouli et al., 2004). The presence of O⁶-mG in the template DNA strand during DNA synthesis will result in an incorporation of a thymine (T) in the nascent strand instead of cytosine (C). The Resultant O⁶-mG-T mismatches activate the MMR pathway. However, having only the ability to repair the un-methylated nascent strand (Marti and Fleck, 2004), activated MMR pathway (involving MutSα and MutLα complexes) will remove the thymine but not the culprit O⁶-mG. This will lead to repeated cycles of O⁶-mG-T mismatches and repeated futile attempts at repair by MMR pathway, ultimately resulting in DNA double-strand breaks activating ATR/Chk1 S-G2 checkpoint pathway which leads to cytotoxic effect (Yoshioka et al., 2006).

Although it is estimated that that O⁶-mG accounts for less than 8% of the lesions caused by TMZ they are responsible for most of the cytotoxicity caused by TMZ (Kaina, 2003). Apart from this, presence of O⁶-mG is postulated to directly induce apoptosis via mechanism that is dependent on MutSα and MutLα (Stojic et al., 2004; Karran, 2001). Moreover, cells with reduced levels of MSH2 and MLH1, in spite of being capable of MMR, fails to activate cell cycle checkpoints or undergo apoptosis in response to alkylating damage (Lettieri et al., 1999; Claij and te, 2004; Cejka et al., 2003). The lack of effective MMR pathway can make cells resistant to alkylating/TMZ therapy (Takagi et al., 2008) (Karran P. (1990) Mutat Res 236: 269-75). These data highlights the need of a functional MMR pathway for the cytotoxicity of TMZ. Previous studies have demonstrated that MMR pathway can be regulated by post-translational modifications such as nuclear translocation of MSH2 and MSH6 in response to TMZ therapy (Christmann M. (2000) J Biol Chem 275: 36256-36262) but not the regulation at gene transcription level.

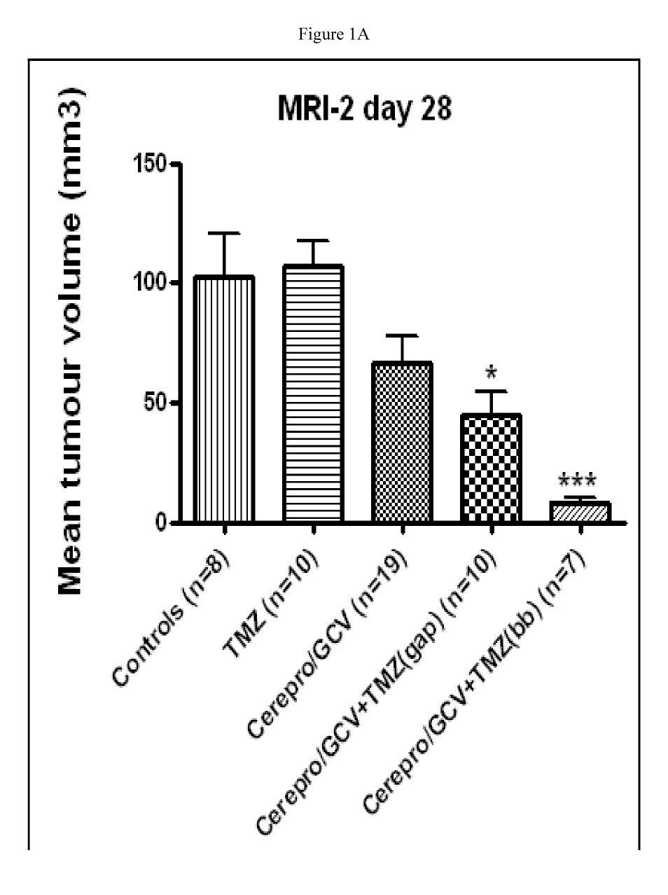

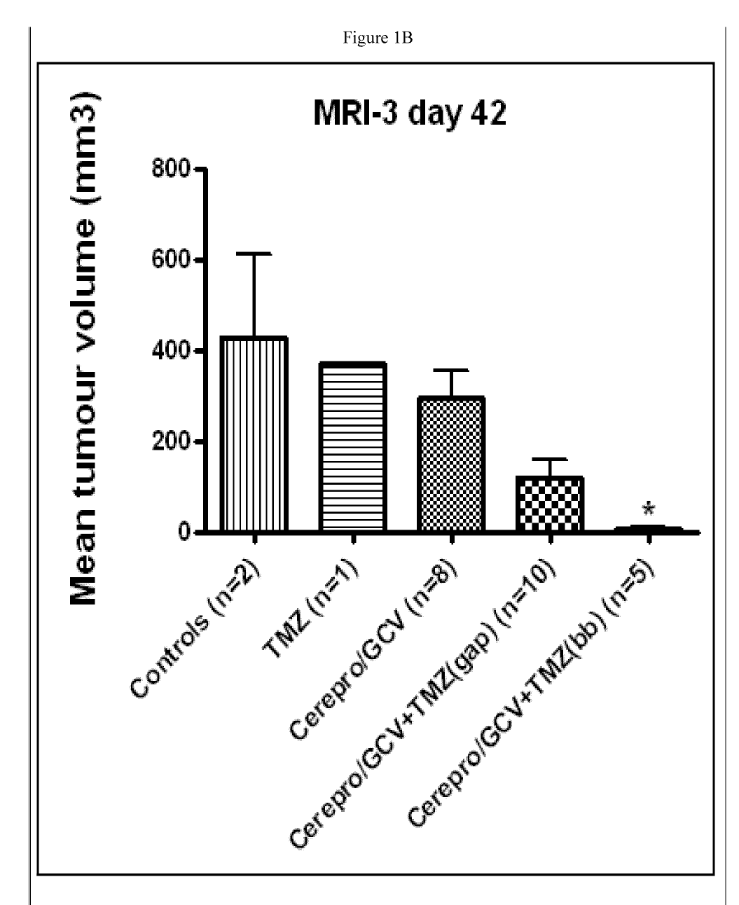

Apart from O⁶-mG other alkyle DNA base adducts and abasic sites (AP) are formed after TMZ therapy. It is known that N7 methyle guanine (N7mG) and N3 methyle adenine (N3 mA) account for respectively 80-85% and 8-18% of methyle adducts formed by TMZ (Reviewed by Kaina et al. (2007) DNA repair 6: 1079-1099). These lesions become important in cytotoxicity if O⁶-mG is repaired by MGMT or cannot act due to the lack of effective MMR pathway. Of these, the N7 mG is stable (t1/2 40-80 h) but not cytotoxic by itself. However, this can be spontaneously depurinated or cleaved by DNA glycosylase (such as methyle purine glycosolase, MPG) that is part of the base excision repair (BER) pathway into AP sites, which are highly toxic to the cells. Repair imbalances caused by over expression of MPG (and/or inhibitin of the BER downstream of MPG such as APE) can result in this type of cytotoxicity (Kaina 2007) (Adhikari 2008). Accumulation of N3 mA is cytotoxic but is readily hydrolysed by MPG into AP, which are cytotoxic if not repaired by BER pathway involving APE (Adhikari 2008) (FIG. 1). This illustrates that imbalances of MPG or downstream BER can still lead to cytotoxicity. However, it must be emphasised that O⁶-mG is the most critical lesion responsible for TMZ induced cytotoxicity. In this experiment we have not studied in-depth the effects of AdHSV-tk/GCV gene therapy on BER pathway mediated cytotoxicity, especially on the key enzymes such as MPG, APE and Pol β, which can affect the cytotoxicity of TMZ, and warrants further evaluation.

Role of AdHSV-Tk/GCV in Enhancing the Cytotoxicity of TMZ

In our study we were unable to demonstrate the presence of MGMT protein by western blot and IHC or the mRNA with RT-RCR, suggesting that the BT4C cell line that we use does not have a functional MGMT repair pathway. Hence, theoretically, the cell line should be sensitive to TMZ therapy. However, both in vitro and in vivo experiments there were no effect with TMZ therapy alone, implicating that other mechanism of resistance are in operation.

The efficacy of TMZ is known to be affected by different DNA repair activities (Kaina, 2003; Kaina et al., 2007; Parkinson et al., 2008; Takagi et al., 2003; Takagi et al., 2008) and by the presence of functional p53 activity (Blough et al. (2010) J Neurooncol.). Our study showed that BT4C cell line is highly resistant to the TMZ treatment and does not respond to the treatment even with very high concentrations (1000 μM). When the cells were first treated with AdHSV-tk/GCV, however, TMZ (in high concentration) started to demonstrate cytotoxicity.

RT-PCR analyses revealed that both MSH-2 and MLH-1 gene expressions were clearly elevated after AdHSV-tk/GCV in all groups that received gene therapy (AdHSV-tk/GCV). Enhancement of MMR pathway is beneficial for the success TMZ treatment. The up-regulation of gene expression of MSH-2 and MLH-1 could be detected already within 96 hours after adding GCV and was observed for several days after initiation GCV. It is possible that the up-regulation persist for an even longer period of time, but this could not be confirmed since the treated cells did not survive beyond this point.

In current treatment protocols used in human clinical trials, TMZ is commenced after completing 14 days of GCV therapy. In most cases, there had been a considerable gap in time between completing GCV therapy and beginning TMZ therapy. This gap was thought helpful, as it enabled the patient to recover somewhat from the physiological stress imposed by GCV, before imposing the added stress of TMZ therapy.

Our results suggest that this gap is in fact an impediment to synergistic benefit. Rather, we have found that TMZ therapy should be commenced as early as possible after completing GCV therapy (while the up-regulation of MMR pathway is still in-place) or more preferably, commenced concomitantly with GCV therapy (where the TMZ therapy is started a few days after starting GCV therapy), giving time for optimal induction of the MMR pathway.

The exact role of MMR pathway in AdHSV-tk/GCV gene therapy is not known but what is known is that MMR deficiency enhances the tumour cell sensitivity to GCV at high GCV concentrations (O'Konek et al., 2009). It is not clear whether activation of MMR pathway by AdHSV-tk/GCV leads to repair of DNA damages caused by the gene therapy. Incorporation of GCV triphosphate into the DNA is known to cause GC to TA transversions, errors in DNA replication and increase the mutation frequency that can contributes to the activation of MMR pathway. It is also possible that generation of GCV triphosphate which competes with dGTP for the incorporation into DNA could lead to dNTP pool imbalance leading to misincorporations resulting in activation of MMR pathway (Martomo and Mathews, 2002; Kunz, 1982; Bebenek et al., 1992).

We acknowledge the fact that both GCV and TMZ have mylelosuppressive adverse effects which can lead to synergistic toxicities. Results from our study also show that TMZ has a profound adverse effect on the WBC and platelet counts in rats. GCV alone or in combination with AdHSV-tk did not reduce these values significantly. In the groups where the treatments were combined the reductions in WBC and platelet counts were not significantly lower compared to the groups that received TMZ only. However, more frequent FBC analyses may be needed before making any conclusions. In the event that synergistic adverse effects occur it would be possible to formulate GCV in a wafer format (like Gliadel wafer) that can be delivered into the tumour cavity after surgery for the local effect, avoiding systemic toxicities and further improving GCV delivery into the tumour.

Relevance for Recurrences

Since the MMR pathway plays a significant role in the cytotoxicity of TMZ, there is selective pressure on the tumour cells to loos the MMR function for their survival. Several studies have shown that resistance to TMZ therapy occurs by loosing MMR function, ultimately leading to recurrences with hypermutation phenotype (Cahill et al., 2007; Hunter et al., 2006). It would be interesting to see whether there is a reduction or a delay in the occurrence of these treatment resistant hypermutator phenotype recurrences when TMZ is combined with AdHSV-tk/GCV therapy, which is known to be more sensitive against cells lacking MMR function (O'Konek et al., 2009). Malignant gliomas recurring after TMZ therapy may be more sensitive to AdHSV-tk/GCV gene therapy due to the same reason and we believe it is worth further evaluation.

Other chemotherapeutic agents dependent on a

Functional MMR Pathway for their Cytotoxicity

Deficiencies in the MMR pathway is known to make cancer cells resistant to several classes of chemotherapeutic agents such as alkylating/methylating agents such as MNNG, procarbazine (Koi et al., 1994; Kat et al., 1993), platinum based chemotherapeutics such as cisplatin (Aebi et al., 1996; Clodfelter et al., 2005; Drummond et al., 1996), doxorubicin (Duckett et al., 1999), epirubacin, mitoxantrone, camptothecin, topotican (Fedier et al. 2001), 5-FU (Meyers et al., 2003) and antimetabolites such as 6-thioguanine (Aquilina et al., 1993) (Table 1). We postulate that up-regulation of the MMR pathway by AdHSV-tk/GCV (and LentiHSV-tk/GCV) could increase the cytotoxicity of these agents in relevant cell culture models. Thus, up-regulation of MMR pathway by AdHSV-tk/GCV gene therapy could make it an ideal combination therapy partner for these chemotherapies by enhancing MMR pathway and the cytotoxicity of these chemotherapies in the treatment of different cancers, preventing or delaying the emergence of treatment resistance and the occurrence of hypermutator phenotype recurrences. High sensitivity of AdHSV-tk/GCV on MMR negative cells (as noted by others) will make it an ideal in the treatment of recurrences following the treatment with those chemotherapies.

How to Make or Use

The present invention requires the administration of a vector having a functional gene, and a prodrug which can be converted by an expression product of that gene, into a cytotoxic agent. Preferably, the functional gene is a functional thymidine kinase gene. Preferably, the prodrug is ganciclovir or its analogues. It will be understood that the prodrug therapy should commence after the vector has been administered.

Alternatively, suicide genes such as cytosine deminase, cytochrome P450, E. coli purine nucleoside phosphorylase and carboxypeptidase G2, are suitable for use in the invention. Those suicide genes can be used in combination with suitable prodrugs, such as 5-fluorocytosine, cyclophosphamide, 6-methylepurine or F-araAMP or 4-benzoyl-L-glutamic acid (CMDA) or their chemical analogs, respectively. In one embodiment, the suicide gene, i.e. the vector, is cytosine deminase, and the prodrug is 5-fluorocytosine is suitable for use in the invention.

The vector is preferably locally administrated. When the therapy is of a cancerous tumour, for example, the vector may be administered directly into that cancerous tumour. Alternatively, it may be preferable to surgically remove the tumour, and then administer the vector into the tumour bed. As used herein, the term “tumour bed” means the tissue that is exposed upon removing a tumor (or part of a tumor). Due to the diffuse nature of brain cancer growth, the tumor bed may contain malignant cells.

Preferably, the tumour resection is complete as possible, i.e. more than 90%, 95% or 98%. In a preferred embodiment, the vector is administered by injection approximately 1 cm (preferably between 0.5 cm and 5 cm, more preferably between 0.8 cm and 3 cm) deep into the wall of the tumour cavity. This ensures that the vector is into healthy tissue, i.e. is targeting primarily healthy cells (although it is appreciated that some malignant cells may reside in that area of apparently healthy tissue).

The vector that is used to transfer the gene may be any viral vector. However, it is preferred that it is derived from an adenovirus or a lentivirus. More preferably, it is derived from adenovirus.

The present invention is a combination therapy, comprising the administration of a gene therapy vector, a prodrug and a cytotoxic agent. The cytotoxic agent is preferably different from the cytotoxic agent that results from conversion of the prodrug (for example conversion of the ganciclovir), but otherwise the exact nature of the cytotoxic agent is not crucial, but it should preferably be a drug whose function is impaired by impaired MMR pathway. Some preferred cytotoxic agents are:

a) a chloroethylating agents such as carmustine, lomustine, fotemustine, nimustine, ranimustine or streptozocin;
b) a non-classical alkylating agent such as procarbazine;
c) a methylating triazine such as temozolomide, dacarbazine, altretamine, or mitobronitol;
d) a DNA cross-linking agent such as cisplatin, carboplatin, nedaplatin, oxaliplatin, triplatin, tetranitrate or satraplatin;
e) a topoisomerase II inhibitor such as doxorubicin, epirubicin, aclarubicin, daunorubicin, idarubicin, amrubicin, pirarubicin, valrubicin or zorubicin, mitoxantrone or pixantrone;
f) a topoisomerase I inhibitor such as topotecan, camptothesin, irinotecan, rubitecan or belotecan;
g) an anti metabolite (pyrmidine analogue) such as 5-FU, capecitabine, tegafur, carmofur, floxuridine or cytarabine;
h) an anti metabolite (purine analogue) such as 6-thioguanine or mercaptopurine; or
i) a cytotoxic DNA alkylating agent.

The most preferred cytotoxic agent is temozolomide (TMZ).

For synergy between vector/prodrug/cytotoxic, it is necessary for the MMR pathway to become upregulated, and therefore administration protocol/dosage regimen is key.

As used herein, “cytotoxic therapy” and “prodrug therapy” means the cytotoxic and prodrug dosage regimens, courses of treatment. Those therapies are for a specified period of time. The vector, however, need only be administered once.

Preferably, the another cytotoxic agent therapy begins no later than 7 days after prodrug therapy has finished. More preferably, the cytotoxic agent therapy begins no later than 6, 5, 4, 3, 2 or 1 day after prodrug therapy has finished. Preferably, the cytotoxic agent therapy begins less than 1 day after prodrug therapy finishes.

For the avoidance of doubt, included within the scope of the invention is both the situation where cytotoxic therapy is started immediately after prodrug therapy has finished, and also the situation where cytotoxic therapy is started before the prodrug therapy has finished (i.e. there is a period of simultaneous administration.

The cytotoxic therapy and the prodrug therapy may be started at the same time. Although, preferably, the cytotoxic agent therapy begins no earlier than 2 days after prodrug therapy begins. This allows for most efficient administration as the cytotoxic and prodrug are only combined once the MMR pathway has been upregulated. This is the most efficient dosage regimen.

Preferably, the prodrug therapy and the another cytotoxic agent therapy overlaps. More preferably, the therapies overlap for at least 3 days. More preferably, they overlap for at least 7, 10, 14 or 18 days.

Preferably, the prodrug therapy lasts for from 10 to 20 days. More preferably, it lasts for from 11 to 19, 12 to 18 or 13 to 17 days. Preferably, it lasts for 14 days.

In a preferred embodiment, the prodrug therapy begins from 2 to 5 days after vector administration (gene transfer). More preferably, the prodrug therapy begins at 5 days after gene transfer.

Preferably, the another cytotoxic therapy should begin at the earliest at 2 days after starting prodrug therapy, and at the latest at 7 days after stopping prodrug therapy.

The another cytotoxic agent therapy should begin no earlier than simultaneously with the commencement of prodrug therapy. It will be appreciated that it is preferred for the another cytotoxic agent therapy to begin no earlier than 2 days after commencement of prodrug therapy.

The up-regulation of the MMR pathway is a general advantage of our invention. Therefore, it will be appreciated that the agent of the invention is useful in the treatment of a number of conditions. Examples of those conditions are cancer, actinic keratosis, pterygium diabetic retinopathy, atherosclerosis, asthma, chronic obstructive pulmonary disease, sarcoidosis, idiopathic pulmonary fibrosis, rheumatoid arthritis, pseudoexfoliation syndrome of the eye and Alzheimer's disease. The most preferred therapy is of cancer. Preferably, the therapy is of a cancerous tumour, such as malignant glioma, or a tumour of the prostate. An agent of the invention may be used in the therapy of a cancer characterised by a normal or an impaired MMR pathway.

In a further preferred embodiment, an agent according to the present invention, when used to treat a cancerous tumour, also includes the administration of radiation. The radiation is preferably administered after the administration of the vector and the prodrug, and radiation therapy preferably starts at the same time as the cytotoxic chemotherapeutic agent (preferably, therapy is simultaneous).

The following Example illustrates the present invention.

### Example 1

A study was conducted concerning tumour growth rate in a rat glioma model. There were 6 patient groups. Details of agents administered and the dosage regimen are shown in Table 1 below.

TABLE 1

| | | | Protocol (d) | | | | | |
|---|---|---|---|---|---|---|---|---|
| | | | Verifi- | Gene | | | | |
| | | | cation by | trans- | | | | |
| Group | | n | MRI | fers | Gap | GCV | Gap | TMZ |
| 1. | Control | 7 | 0 | — | — | — | — | — |
| 2. | TMZ | 10 | 0 | — | — | — | — | 5-9 |
| 3. | AdHSV-tk + | 18 | 0 | 1 | 4 | 5-11 | — | — |
| | GCV | | | | | | | |
| 4. | AdHSV-tk + | 18 | 0 | 1 | 4 | 5-11 | 5 | 17-21 |
| | GCV + TMZ | | | | | | | |
| | (gap) | | | | | | | |
| 5. | AdHSV-tk + | 7 | 0 | 1 | — | 2-9 | — | 9-13 |
| | GCV + TMZ | | | | | | | |
| | (bb) | | | | | | | |
| 6. | AdHSV-tk + | 7 | 0 | 1 | 4 | 5-18 | — | 14-18 |
| | GCV + TMZ | | | | | | | |
| | (sim) | | | | | | | |

The results are shown in FIGS. 1A and 1B. As shown on those Figures, Group 5 showed the largest decrease in tumour size.

### Example 2

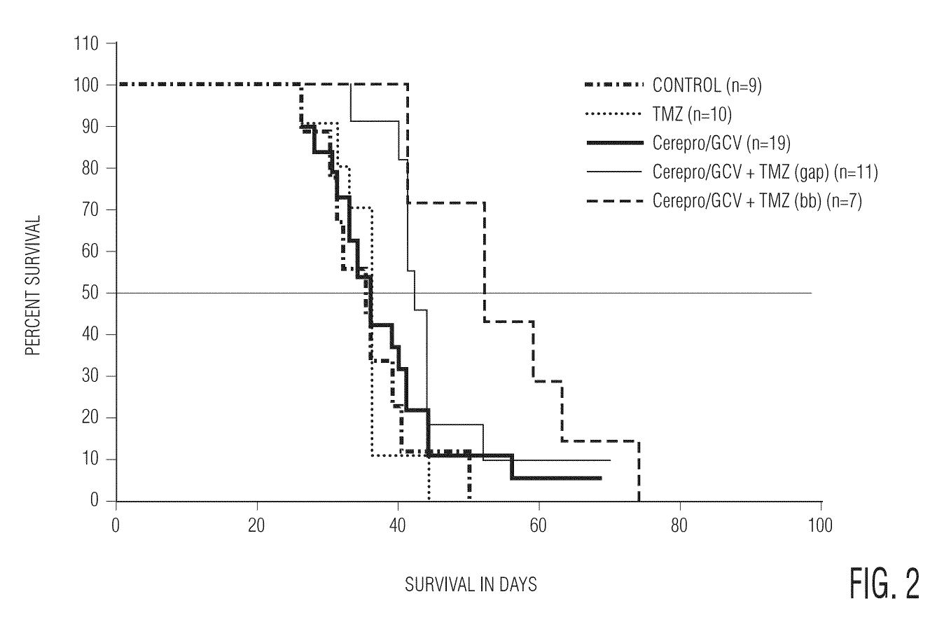

We conducted a second study in the rat glioma model concerning survival rates. Our results are shown here in FIG. 2.

FIG. 2 shows that at 50 days, only 18% (2 of 11) of AdHSV-tk/GCV+TMZ “gap” subjects survived (red line), while 71% (5 of 7) of AdHSV-tk/GCV+TMZ “bb” (no gap) subjects survived (green line). This shows that eliminating the delay between AdHSV-tk/GCV and TMZ is critical, and produces synergistically beneficial results.

FIG. 2 also shows that the AdHSV-tk/GCV+TMZ “bb” (no gap) group had the longest average survival time, and the longest maximum survival time. This confirms that our dosage regimen, with a slight overlap of pro-drug/cytotoxic therapy, is synergistically beneficial.

### SUMMARY

The skilled artisan may extrapolate from our disclosure to make alternatives to the specific examples we disclose here. For example, the efficacy of many other chemotherapeutic agents are reduced by the impaired MMR pathway. Hence one may explore the possibility of enhancing the cytotoxicity of those chemotherapies by increasing the MMR pathway activity by AdHSV-tk/GCV gene therapy.

Similarly, one may explore the possibility of enhancing MMR pathway by LentiHSV-tk/GCV and to see if this could be utilised to increase the cytotoxicity of chemotherapeutic agents.

Furthermore, treatment with TMZ gives a selective pressure for the cancer cell to lose the MMR function, giving rise to hypermutator phenotype recurrences that have lost MMR function and are resistant to most of the chemotherapeutic agents. HSV-tk/GCV gene therapy is especially effective in killing cells with defective MMR pathways. Taking this into consideration it would be worthwhile to explore whether the occurrence of MMR negative hypermutator phenotype recurrences are either prevented or delayed by combining these chemotherapeutic agents with AdHSV-tk/GCV (and LentiHSV-tk/GCV). Moreover, since TMZ is not effective in recurrent malignant gliomas, especially in the hypermutator phenotype recurrences, our invention provides the first way to make TMZ effective in recurrent malignant gliomas, especially the recurrences after TMZ therapy.

Thus, we intend the coverage of our patent to be defined not by the specific examples we discuss here, but by the legal claims we append here.

We claim:

1. In a method of treating brain cancer in an immunocompetent human patient by administering temozolomide to said immunocompetent human patient, the improvement comprising: administering to said immunocompetent human patient a viral vector.

2. The method of claim 1, wherein said viral vector is administered in an amount of about 1 to 3×10³ cfu.

3. The method of claim 1, further comprising: resecting at least part of said brain cancer.

4. The method of claim 3, wherein said resecting forms a cavity and wherein said cavity has a cavity wall, and wherein said administration of said viral vector comprises administration to the wall of the cavity formed by the resecting.

5. The method of claim 1, wherein said viral vector comprises virus selected from the group consisting of: lentivirus and adenovirus.

6. The method of claim 1, wherein said viral vector comprises a thymidine kinase transgene, and wherein said method of treatment further comprises administering to said human patient ganciclovir or an analogue thereof.

7. The method of claim 1, wherein said brain cancer is selected from the group consisting of: glioblastoma multiforme and anaplastic astrocytoma.

8. The method of claim 1, further comprising administering to said human patient radiotherapy.

9. The method of claim 6, wherein said administration of said ganciclovir or analogue thereof lasts for from about 10 to about 20 days.

10. The method of claim 6, wherein said administration of said temozolomide lasts for not more than about 50 days.

11. The method of claim 10, wherein said administration of temozolomide begins not earlier than about 2 days after said administration of ganciclovir or an analogue thereof.

12. The method of claim 6, wherein said administration of said temozolomide begins not later than about 7 days after said administration of said ganciclovir or analogue thereof.

13. The method of claim 12, wherein said administration of said temozolomide overlaps said administration of said ganciclovir or analogue thereof.

14. The method of claim 13, wherein said overlap is for at least 3 days.

15. A kit comprising a viral vector and temozolomide, said viral vector and said temozolomide present in an amount effective to treat brain cancer in a human patient.

16. The kit of claim 15, wherein said viral vector comprises a thymidine kinase transgene.
